(19) United States
(12) Patent Application Publication
(10) Pub. No.: US 2023/0069992 A1
(43) Pub. Date: Mar. 9, 2023

(54) NOZZLE FOR SPRAYING LIQUID IN THE FORM OF MIST

(71) Applicant: Eveon, Montbonnot-Saint-Martin (FR)

(72) Inventors: Claire Authesserre, Montbonnot-Saint-Martin (FR); Mahutin Akle, Montbonnot-Saint-Martin (FR)

(73) Assignee: Eveon, Montbonnot-Saint-Martin (FR)

(21) Appl. No.: 17/795,782

(22) PCT Filed: Feb. 4, 2021
(86) PCT No.: PCT/FR2021/050208

§ 371 Date: Jul. 27, 2022

(30) Foreign Application Priority Data
Feb. 4, 2022 (FR) 2001108

(51) Int. Cl. B05B 1/34

(52) U.S. Cl. CPC B05B 1/3436; B05B 1/3426; B05B 1/3478

(57) ABSTRACT

The invention relates to a fluid spray nozzle which is intended to be mounted on a distribution receptacle, the nozzle comprising at least one fluid inlet capillary extending longitudinally along an axis, a supply means to receive the fluid from the at least one inlet capillary in order to supply it to a pillar comprising at least two pipes, the pipes extending longitudinally along the axis A1 and being radially offset relative to the axis, at least two turbulence channels in fluid connection with the at least two pipes, a turbulence chamber for receiving the fluid coming tangentially from the at least two turbulence channels in order to supply at least one spray opening having axial symmetry and a constant cross-section s, the chamber having a cross-section decreasing towards the opening and having a maximum cross-section S and a maximum diameter, the ratio of the cross-section s of the spray opening to the maximum cross-section S of the turbulence chamber is 1%≤s/S≤20%.

### FIELD OF THE INVENTION

This invention relates to a nozzle for a device to spray a fluid in mist form. The device is manually or automatically operated using a mechanical system such as a pump, syringe pump, spring, or electromechanical system, i.e., using a motor, to spray the fluid.

### PRIOR ART

It is known to anyone skilled in the art that as viscosity increases, any fluid tends to form large droplets when sprayed. This results in a heterogeneous spray and not a spray that generates a homogeneous mist. This results in wastage of the sprayed product and non-uniform application of the product. It has been shown that the viscosity of a medicinal fluid has a positive influence on the absorption of the drug, hence the need to be able to spray this type of fluid correctly. The nozzles of the prior art have limited capabilities at low viscosities. The nozzles of the prior art are thus not satisfactory when it comes to combining the ability to spray viscous fluids for medical applications, in particular, by means of a system called “airless”, i.e., without propellant.

Indeed, a solution to circumvent this technical problem, is the use of propellant. However, this solution has proven to be unsatisfactory when it comes to ejecting certain pharmaceutical products for sterility reasons, for example. Indeed, the presence of a propellant gas can affect the sterility, cleanliness, or microbiological balance of the area of administration. The environmental impacts of such gases must also be considered.

A propellant-free misting solution also ensures uniform coverage of the sprayer's target area, all with less volume of the fluid sprayed, resulting in cost savings. by furthermore, the spray in mist form has a second advantage concerning the comfort of the patient during administration upon sensitive or painful areas.

Currently, there are piezoelectric misting solutions that are capable of forming a mist. However, these solutions remain relatively limited in terms of the flow rate ranges available for use, the responsiveness of the system—i.e., the speed of administration—and the control of the direction of the mist. In addition, these systems impose a high final cost of the device which can be a limiting factor to the deployment of such a solution, especially in disposable systems.

Application EP2570190, for example, relates to a spray nozzle for dispensing a fluid comprising a fluid chamber for receiving the fluid, at least one supply channel for feeding the fluid from the fluid chamber radially inward into a turbulence chamber, and an outlet channel with an inlet end facing the turbulence chamber and an outlet end for discharging the fluid through the environment of the spray nozzle. The outlet channel of this invention narrows in the direction of the fluid flow. This disclosure further relates to a sprayer comprising such a spray nozzle. This prior art represents an insufficient advance for highly viscous fluids.

Indeed, it is the difference in the speed between the gas, often the air, and the fluid that makes spraying possible. This type of technology, which is perfectly suited for low viscosity fluids, becomes inoperative for fluids exceeding 100 centipoises (cps). In addition, the use of a propellant gas such as air may become problematic when it comes to spraying health, nutrition, or dermo-cosmetic products, since the requirement for sterility must be respected, especially in the medical field.

Application EP0412524 discloses a disposable nozzle adapter for intranasal administration of a viscous medical solution in combination with a spray container, which comprises a cylindrical body, a stem placed within the body and a nozzle tip. The body has a cylindrical chamber and a central bore that communicates with the chamber through a channel for attachment of the spray container. The stem is equipped with at least one small and one medium-sized portion at its end. The nozzle tip has an upper wall and a cylindrical portion extending therefrom, the upper wall being provided with a central spray aperture comprising a tapered recess and turbulence grooves extending outwardly from the tapered recess at the inner surface of the cylindrical portion. The turbulence grooves of this invention have an outwardly increasing cross-sectional area and its cross-sectional area is at least 0.03 to 0.08 mm². The nozzle tip is fitted into the opening of the body chamber and engaged with the medium sized portion of the stem to form an annular channel surrounding the small sized portion of the stem in communication with the grooves. This prior art also represents an insufficient advance for highly viscous fluids.

The subject-matter of this invention is therefore to overcome the disadvantages of the prior art and to improve the ability of the nozzles to spray a viscous rheofluidifying fluid in mist form without propellant. To this end, the purpose of this invention is a nozzle that is free of air or any other propellant that will make the generation of a mist possible from a highly viscous and rheofluidifying fluid that flows at a viscosity of more than 3000 Pa·s at 0.01 s⁻¹, and at a very low flow rate, i.e., a flow rate preferably comprised between 0.10 ml/s and 1 ml/s. This makes it possible to deposit a viscous fluid in a thin layer and in small quantities on a large surface.

### Abstract

The invention relates to a fluid spray nozzle for mounting upon a dispensing container, said nozzle comprising:
at least one fluid inlet capillary extending longitudinally along an axis A1,
a turbulence chamber for receiving the fluid to be sprayed. The turbulence chamber has a maximum cross-section S and a maximum diameter D,

at least two conduits that extend longitudinally along the axis A1 and being radially offset from said axis A1, said conduits in fluid connection with the inlet capillary,
at least two turbulence channels, in fluid connection with said at least two conduits, and connecting said at least two conduits with the turbulence chamber, said at least two conduits thereby connect the inlet capillary to the at least two turbulence channels,
a spray orifice fed by the turbulence chamber. The spray orifice has axial symmetry and a constant cross-sections. Along axis A1, said turbulence chamber has a cross-section that decreases towards said spray orifice. The nozzle is characterized in that:
the ratio of the cross-sectional area s of the spray orifice to the maximum cross-sectional area S of the turbulence chamber is such that 1%≤^{S}/S≤20%, and
the spray nozzle is operated by means of an actuator independent of the nozzle, and
the inlet capillary has a cross-sectional area that allows a fluid shear rate greater than 5000 s⁻¹

Thus, this solution achieves the above-mentioned objective. In particular, production of a homogeneous mist from a viscous rheofluidifying fluid is made possible.

The nozzle according to the invention may comprise one or more of the following characteristics, taken alone or in combination with each other:
1%≤S/S≤10%,
the spray orifice has a cylindrical shape with a diameter d and a height h such that: 40% d≤h≤150% d, preferably 50% d≤h≤100% d,
the at least two turbulence channels each have a right-angle quadrilateral cross-section. Said cross-section is between 0.001 and 0.06 mm²,
the quadrilateral is a square,
the supply means comprises:
   either a hollow section chamber of generally cylindrical shape, the base of which extends along a plane perpendicular to the axis A1,
   or several supply channels that extend radially on a plane perpendicular to the axis A1,
   in order to supply said at least two conduits,
the turbulence chamber has a truncated cone shape whose angle α between the axis A1 and the generatrix is such that 25°≤α≤55°, preferably 30°≤α≤45°,
the at least two conduits are formed in a pillar. Said pillar comprises a jacket cylinder that has an inner surface. Said jacket cylinder comprises a coaxial spacer whose outer surface is polygonal so that the edges of the spacer contact the inner surface of the jacket cylinder thereby forming at least three pillar conduits,
the at least one inlet capillary comprises at least two portions each with a constant diameter along its length, each portion having a diameter equal to or greater than the diameter of at least one downstream portion and each portion having a diameter equal to or less than the diameter of at least one upstream portion.

The invention also relates to a medical device capable of dispensing a fluid and comprising a nozzle according to any of the preceding claims.

The invention also has as its subject matter a method of dispensing a rheofluidifying viscous fluid by spraying, characterized in that the method is carried out by means of the nozzle according to any of the above characteristics.

According to this process, the distribution may be carried out in mist form with homogeneous drops. At least 90% of the mist's drops have a diameter of less than 100 μm. The distribution may also be carried out in the form of a mist with homogeneous drops whose median diameter is between 10 μm and 50 μm. The distribution may also be realized in the form of a mist with homogeneous drops of which less than 12% of the drops have a diameter of less than 10 μm. Finally, the distribution may be performed as a homogeneous mist with a droplet dispersion characterized by the deviation ratio of Dv10 and Dv90 from the median, which is less than 2.

### Definitions

In this invention, the following terms are defined as follows:
“Upstream” is defined according to the direction of fluid flow through the nozzle, and refers to any element that is located, relative to another element, near the fluid inlet in the nozzle.
“Downstream” is defined according to the direction of the fluid flow through the nozzle, and designates any element that is located, relative to another element, near the fluid outlet of the nozzle.
“Mist” is similar to a mist and is defined as a mass of very fine droplets.
“Capillary” is a conduit with a section that is thin relative to its length. The section is unspecified.
“Pillar” is an element composed of one or more parts which, when assembled, serves as a support for at least the supply means and the conduits, in the context of the invention. The pillar is located between the supply means and the turbulence channels. It comprises at least the means for conveying the fluid from at least one capillary to the channels.
“Turbulence channel length”: the length of the turbulence channels is defined as the longest distance with identical sections along said channels.
“Viscous fluid”: fluid whose viscosity is higher than 10 mPa·s.
“Rheofluidizing fluid”: a fluid with a dynamic viscosity that decreases as the shear rate of the fluid increases.

“Dv10, Dv50, Dv90” are quantities used in granulometry that give an indication of the volume distribution of the particle size of a set of particles (in this case, droplets). A Dv10 of 4 μm indicates that 10% of the particles (by volume) are less than 4 μm in diameter. D50 gives the median size: half of the particles are smaller, half larger, and 10% of the particles are larger than D90. In other words: Dv10, Dv50 and Dv90 indicate particle sizes for which 10%, 50% and 90% (respectively) of the particle population are smaller than this size.
“Distribution” or “SPAN” is the distribution around the median, or Dv50 of different drop sizes measured in a mist. It is obtained by the ratio of the difference between the Dv10 and Dv90, and the Dv50. This ratio is unitless.

### BRIEF DESCRIPTION OF THE FIGURES

FIG. 1 is an illustrative view of the spray that the invention wishes to avoid on the left (a) and the desired mist spray on the right (b).

FIG. 2 is a front view of an embodiment according to the invention with three cross sections (2a, 2b, and 2c).

FIG. 3 is an isolated front view of FIG. 2 showing the orifice and base sections of a truncated cone-shaped turbulence chamber.

FIG. 4 is a perspective view of the fluid path within the nozzle, the pillar is transparent.

FIG. 5 is a perspective view of the fluid path of a nozzle according to another embodiment of the invention in which the supply means is composed of a plurality of angularly spaced channels.

FIG. 6 consists of three FIGS. 6a, 6b, 6c) illustrating three different embodiments for the inlet capillary, this figure illustrates the fluid paths.

FIG. 7 is a perspective view of the fluid path within the nozzle according to an embodiment where the conduits are formed by a spacer inserted into the pillar cylinder, here shown in transparency.

FIG. 8 is a cross section of the pillar to illustrate the conduits between the spacer and the enveloping cylinder.

FIG. 9 is a view of two embodiments according to the invention in which the length of the conduits has been changed from H1 to H2.

FIG. 10 shows the logarithmic relationship between viscosity and shear for a rheofluidizing fluid suitable for spraying through the nozzle according to the invention.

FIG. 11 illustrates a cross-sectional view of the nozzle according to the invention with a clearance for connecting a container holding the fluid to be expelled.

FIG. 12 illustrates a perspective view of the spacer of one particular embodiment according to the invention having multiple parallel inlet capillaries.

FIG. 13 shows a perspective view of the resulting fluid path with multiple inlet capillaries as in FIG. 12.

FIG. 14 is a schematic view of the nozzle according to the invention where the pillar is confused with the part forming the turbulence chamber, the orifice, and the turbulence channels.

### DETAILED DESCRIPTION

The following description will be better understood from the drawings shown above. For the purpose of illustration, the nozzle is shown in preferred embodiments. It should be understood, however, that this application is not limited to the specific arrangements, structures, characteristics, embodiments, and appearances indicated. The drawings are not drawn to scale and are not intended to limit the scope of the claims to the embodiments shown therein.

In general, the invention relates to a spray nozzle 1 for fluid, more specifically for a viscous, rheofluidifying fluid, to be mounted upon a dispensing container.

The fluid considered may not be a rheofluidizing fluid if its viscosity is of the order of 20 mPa·s, preferably less than 20 mPa·s, i.e., if the viscosity of the fluid is low.

Therefore, nozzle 1 according to the invention is designed to be attached to a fluid reservoir, specifically, a rheofluidifying viscous fluid.

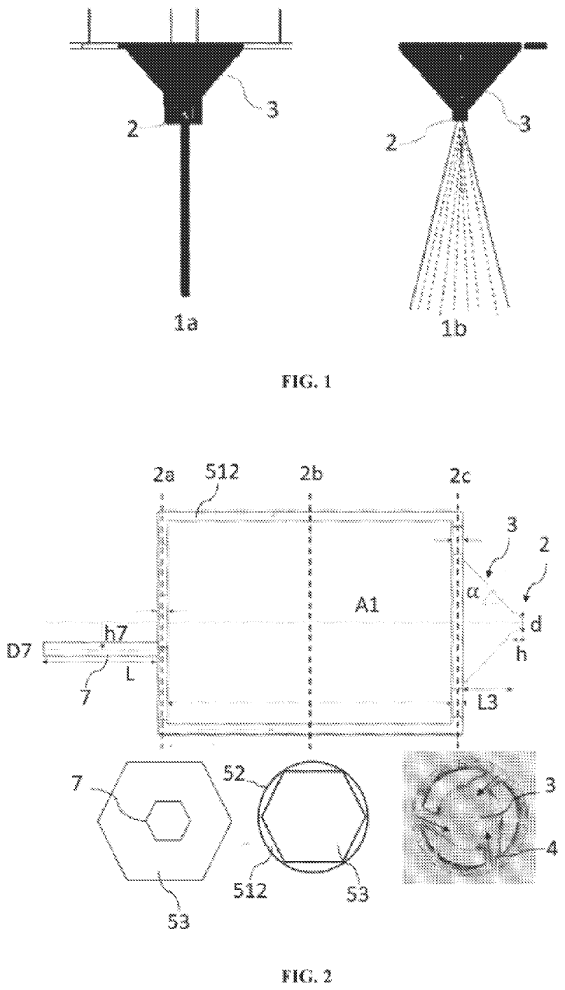

FIG. 1 compares a diffuse mist obtained with nozzle 1 according to the invention, with what is obtained if all the conditions are not met, i.e., an expulsion of a large volume of liquid on a very localized surface.

In FIG. 1, the turbulence chambers 3 and spray orifices 2 of nozzle 1 are illustrated to show the dimensional differences. FIG. 1 is not shown at full scale of the invention. The FIG. 1 is an illustrative view of the spray that the invention wishes to avoid on the left (a) and the desired mist spray on the right (b). In addition, we also want to avoid coarse drops.

In FIG. 2, from left to right, cross sections 2a, 2b, and 2c show the various elements for a better understanding of nozzle 1 according to the invention.

FIG. 2 is a front view of the embodiment of nozzle 1 of the invention. In this figure, inlet capillary 7 of nozzle 1 is offset with respect to axis A1 of spray orifice 2. Axis A1 is also the axis of the truncated cone-shaped turbulence chamber 3. The offset may range from a distance h7 between 0 mm and 0.4 mm, noting that if h7 is equal to 0 mm, this leads to co-axiality with the spray orifice. Distance h7 thus qualifies the distance between the axis A1 of spray orifice 2 and the axis of inlet capillary 7. The advantage presented by this offset is a practical advantage of the embodiment of nozzle 1.

Length L and the cross-sectional area D of inlet capillary 7 are variables that may be acted upon in the context of the invention in order to modulate the shear rate of the fluid passing through inlet capillary 7. In the particular case where cross-section D of inlet capillary 7 is a disk (the capillary being cylindrical), then the cross-section becomes diameter D, such that S=pi*(D/2)², where S denotes the cross-section.

As is well known, the shear rate increases as the cross-sectional area S decreases. At a given flow rate, increasing length L increases the time the fluid is sheared at a given shear rate. This makes it possible to ensure that length L is greater than the flow establishment length and that the viscosity to be achieved at this shear rate is attained. However, the objective is to also reduce the inlet pressure of nozzle 1 and thus to reduce the pressure losses within it. Pressure losses increase as the cross-sections decrease and the lengths increase. It is therefore a question of finding a functional balance, which is achieved by this invention.

In the embodiment shown in FIG. 2, inlet capillary 7 has a cylindrical shape with a circular cross section. Preferably diameter D7 of inlet capillary 7 is between 0.1 and 0.3 mm and its length L is between 2 and 11 mm.

Those skilled in the art know that reducing cross-section D of inlet capillary 7 increases the shear rate of the fluid to be propelled, since the shear rate is equal to the fluid velocity divided by the air gap. This leads to a decrease in the viscosity of said fluid within inlet capillary 7 and in nozzle 1 in general.

Since the viscosity is lower, it is possible to increase the flow rate and thus achieve higher flow velocities while remaining at relatively low pressures. Indeed, at constant viscosity, if we increase the flow rate, we increase the pressure, and this is all the more important as the viscosity is high. In other words, increasing the shear rate reduces the viscosity and thus allows higher flow velocities to be achieved without increasing (very significantly) the pressure. Increasing the speed makes it possible to reach the critical speed which permits generation of an atomization of the fluid and thus create a spray (or mist).

In other words, by significantly shearing the fluid as soon as it enters nozzle 1 and thus as soon as it enters inlet capillary 7, a low viscosity is achieved along the entire fluid path. This makes it possible to achieve a high flow rate and velocity of the fluid, allowing the atomization of the fluid at the nozzle outlet, i.e., the formation of a spray (mist), without very significantly increasing the pressure at nozzle inlet 1. In other words, it makes it possible to produce a spray (mist) from a rheofluidizing viscous fluid at low pressure, thus facilitating the design of a medical device and limiting risks for its user.

The small cross-sections make high velocity possible, however a very small cross-section induces a great drop in pressure, and thus requires that very high pressure be applied at the inlet of nozzle 1 to attain the spray.

In the embodiment shown in FIG. 5, one notes that inlet capillary 7 presents portions 71, 72, 73, 74 from upstream to downstream with different diameters, each portion 71, 72, 73, 74 presents a constant section over its entire length, however, the first portion 71, located the most upstream from inlet capillary 7, presents diameter D as higher than that of downstream portions 72, 73, 74. Each portion 71, 72, 73, 74 thus has, over its entire length, diameter D
that is greater than or equal to that of downstream portions 72, 72, 74, and
less than or equal to that of portions 71, 72, 73 located upstream. The objective here is to progressively reduce the cross-sectional area to increase the shear rate of the fluid in order to reduce the viscosity of the fluid, without creating excessive point restrictions that would induce significant singular pressure losses, and therefore an increase in pressure.

In other words, the closer a portion 71, 72, 73, 74 of inlet capillary 7 is located to turbulence chamber 3, the smaller its cross-sectional area D is. The different positions 71, 72, 73, 74 may be separated from each other by trays. These plates make a better alignment between the different portions 71, 72, 73, 74 possible.

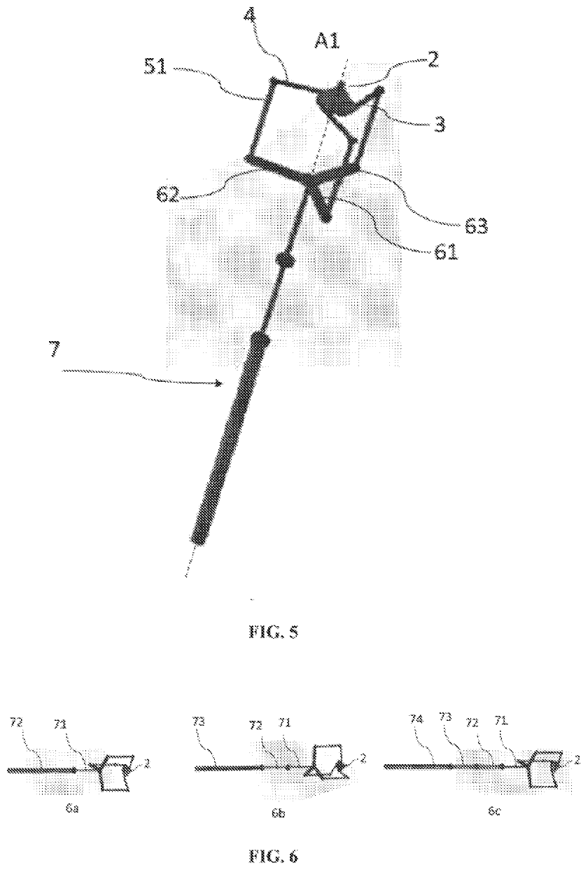

The three variants 6a, 6b and 6c in FIG. 6 illustrate different possible configurations for different portions 71, 72, 73, 74 of inlet capillary 7. According to variant 6a, there are two portions 71 and 72 each with a constant diameter D along its length. Diameter D of downstream portion 71 is smaller than diameter D of upstream portion 72.

Variant 6b, has three portions 71, 72 and 73, each with a constant diameter D along its entire length. Diameter D of the upstream portion 73 is larger than that of central portion 72, itself larger than that of downstream portion 71. This is the embodiment shown in FIG. 5.

Variant 6c, on the other hand, has four portions 71, 72, 73 and 74, each with a constant diameter D. Diameters D are decreasing towards supply means 6 and the two central portions 72 and 73 have similar surface sections. This increases the length of the central portion 72, 73 of the intermediate section. The advantage of this design is to increase length L of inlet capillary 7 when there is only one capillary diameter (flow establishment length at this shear). It is also preferable to increase the length on the intermediate section rather than on the smallest section in order to not increase the pressure losses too much.

In the three embodiments shown in FIG. 6, portions 71, 72, 73, 74 of inlet capillary 7 are coaxial, along axis A1, with spray orifice 2.

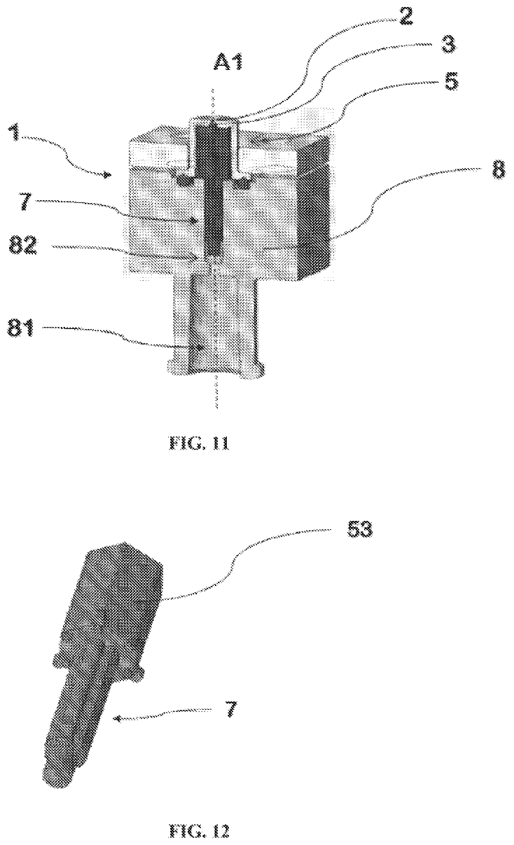

FIG. 11 illustrates a perspective view of nozzle 1 of one particular embodiment according to the invention that has multiple parallel inlet capillaries. The advantage of having several parallel inlet capillaries 7 is how easy it makes industrial manufacturing. In plastic injection molding, it is not possible to make an inlet capillary 7 with a small cross-section, but due to this assembly, it becomes possible to make a cylinder with a much larger diameter (feasible in plastic injection molding) into which the inlet cylinder of nozzle 1 is inserted. This is also feasible in plastic injection molding. In this embodiment, nozzle 1 comprises a support 8 having a first opening 81 suitable for receiving a container containing the fluid to be expelled, and a second opening 82 suitable for receiving a nozzle inlet cylinder. This nozzle inlet cylinder is shown in FIG. 12. In the illustrated embodiment, this nozzle inlet cylinder is supplied with a spacer 53 whose function will be explained later in the application. Grooves are provided longitudinally in the nozzle inlet cylinder, so that the outer walls of the nozzle inlet cylinder can, by interlocking with the inner walls of the second recess 82, form inlet capillaries 7 parallel to each other and extending along the axis A1. Thus, each inlet capillary 7 is formed by a space between the inlet cylinder of nozzle 1 and its support 8. The advantage of support 8, is that nozzle 1 may be connected directly to a syringe via a luer connection (82 is a female luer, the syringe ends in a male luer). This method allows the desired shear rate to be attained at the inlet of nozzle 1 with parts that may be manufactured by industrial production methods (large series).

Generally speaking, the fluid inlet capillary(ies) 7 have a diameter D to generate a fluid shear rate greater than 5000 s⁻¹. In the case of the embodiments with an inlet capillary 7 with variable portions 71, 72, 73, and 74, it is the upstream portion 74 that makes it possible to attain a shear rate greater than 5000 s⁻¹. The following sections 71, 72, 73 allow even higher shear rates to be attained.

The section along axis 2a of FIG. 2 shows the connection that permits the fluid path between inlet capillary 7 and the spacer 53 mentioned above. The spacer 53 in embodiment 2a is a hexagonal prism. Advantageously, this shape makes the creation of conduits with small passage sections possible by using two interlocking parts that can be easily assembled and positioned. It is the gap between the enveloping cylinder and the spacer 53 that makes it possible to form the conduits. The small cross-sectional area makes it possible to maintain a high shear rate.

More generally, inlet capillary 7 is connected to the truncated cone-shaped turbulence chamber 3 by means of conduits 512. These conduits 512 may be obtained in various ways. One way to obtain these conduits 512 is to stack machined parts, thus forming a pillar 5 in which said conduits 512 are provided. However, this method is long and tedious, and industrially unattractive. Alternatively, in the embodiment shown in FIG. 2, these conduits 512 are obtained by means of interlocking two parts that may be obtained independently of each other by plastic injection. These two parts take the form of a hexagonal prism spacer 53 and an enveloping cylinder 52. This limits the number of parts to two, simplifying the assembly of nozzle 1. In the embodiment shown in FIG. 2, spacer 53 and the enveloping cylinder form a pillar 5. Thus the cross-section along axis 2b shows the connection allowing the fluid path between inlet capillary 7 and turbulence chamber 3 through pillar 5. Specifically, section 2b shows the 6 small cross-sectional flow channels formed by interlocking of spacer 53 within enveloping cylinder 52. In this example, spacer 53 is hexagonal, forming six conduits 512, however some embodiments have twelve conduits 512. The greater the number of “facets” in the spacer 53, the smaller the cross-sectional area of conduits 512 and thus, the greater the shear rate. In particular, the fluid path passes between the outer walls of spacer 53 and the inner wall(s) of enveloping cylinder 52. In this example, the enveloping cylinder 52 is circular in cross section. The conduits 512 extend longitudinally along the axis A1. The arrows indicate the direction of the flow from a sprayable fluid along conduits 512 of pillar 5.

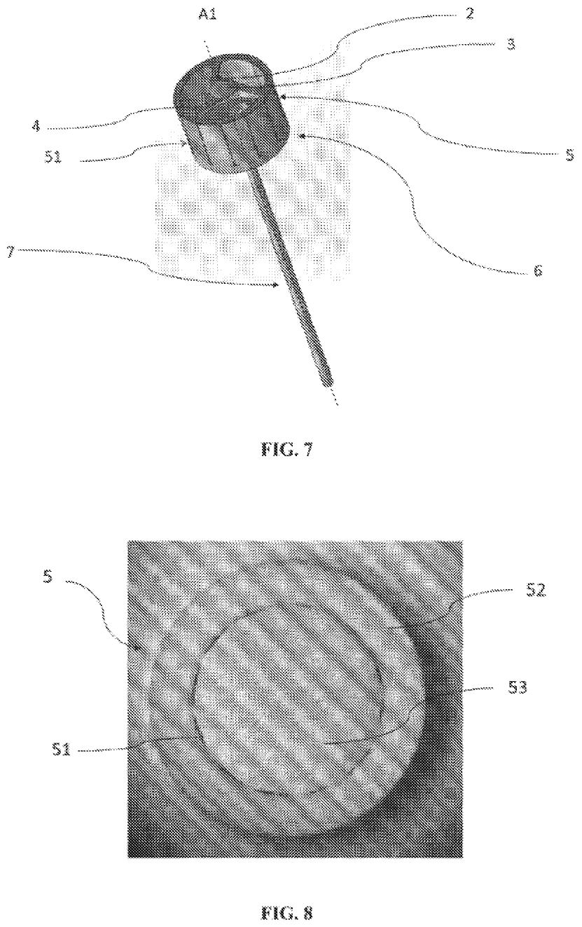

FIG. 7 illustrates the fluid path of a third embodiment according to the invention. In fact, in FIG. 7, pillar 5 is not shown in order to show the fluid path passing through the conduits 51 which extend longitudinally along the axis A1. In this termination mode, all the elements and their dimensions given for the first embodiment are identical except for pillar 5 and its components. There is a series of 12 conduits 51 equally spaced around axis A1, along the circumference of pillar 5. These conduits 51 have a generally flattened shape, resulting from a “faceted” spacer 53 in a cylinder. As before, the greater the number of “facets” in spacer 53, the lower the passage section of conduits 51 and therefore the greater the shear rate within conduits 51. This allows a high shear rate to be maintained to keep viscosity low; the shear rate can be higher than in the fluid path upstream of these conduits 51, allowing for further rheofluidification.

FIG. 8 shows the conduits 51 defined by the space between the surfaces of the spacer 53 and the inner surface of enveloping cylinder 52. Spacer 53 is composed of 12 surfaces forming as many conduits to convey the fluid to be sprayed from the supply means 6 to the turbulence channels 4.

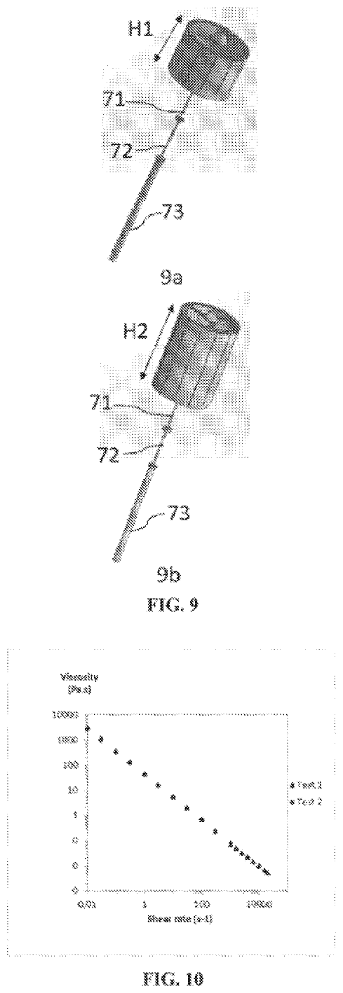

FIG. 9 illustrates two embodiments 9a and 9b in which heights H1 and H2 of pillar 5 are variable to provide a longer length over which the fluid is sheared.

The advantage of height H1 compared to height H2 is that the shorter lengths induce a lower pressure drop and therefore a lower pressure at nozzle inlet 1. The advantage of height H2 over height H1 is that the length over which the fluid is sheared is greater and therefore induces better shear. Here again, a compromise must be made, which is the subject-matter of this invention.

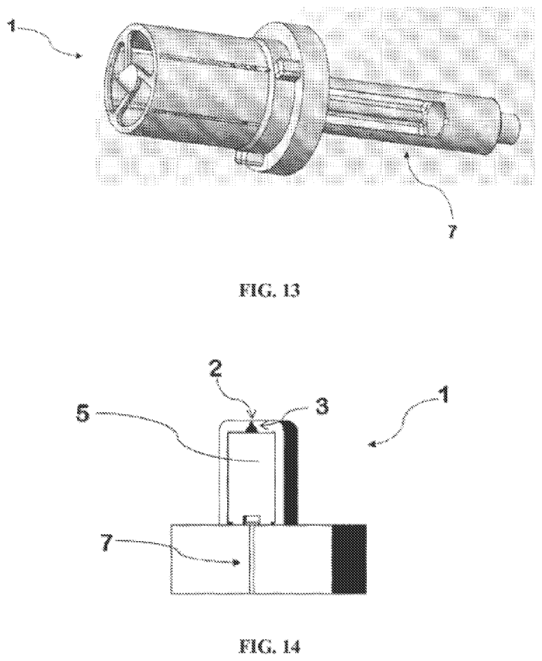

FIG. 14 is another perspective view of a nozzle 1 according to the invention showing an inlet capillary 7 through which the fluid to be expelled will pass. An embodiment with multiple inlet capillaries 7 as shown in FIG. 13 is possible. Also shown is pillar 5, which includes conduits defined by the space between the surfaces of the spacer 53 and the inner surface of the enveloping cylinder 52 (not shown). On leaving the conduits (not shown), the fluid passes through the turbulence channels (not shown) and then tangentially into turbulence chamber 3 before being expelled through spray orifice 2 as a mist. In this embodiment, pillar 5 is merged with the part in which the channels, cone, and spray orifice are formed. In other words, in this embodiment, a single part supports the enveloping cylinder, turbulence channels 4, turbulence chamber 3 and orifice 2.

The connection between conduits 512 of pillar 5 and inlet capillary 7 may be provided by a supply means 6 typically taking the form of a hollow tray of a generally flat cylindrical shape. This is illustrated in FIG. 4. FIG. 4 illustrates the fluid path followed by the fluid to be sprayed into nozzle 1. In the embodiment shown in FIG. 4, nozzle 1 has three conduits 511, 512, 513 connecting inlet capillary 7 to turbulence chamber 3. In this embodiment, these three conduits 511, 512 and 513 are cylindrical shaped conduits with a circular cross section extending longitudinally along the axis A1. The three conduits 511, 512 and 513 are angularly equidistant and thus 120° apart.

The section along axis 2c of FIG. 2 shows more particularly the connection continuing the fluid path between pillar 5 and truncated cone-shaped turbulence chamber 3. The cross-section in FIG. 2c shows a circular ring connecting pillar 5 to the turbulence channels 4 in order to convey the fluid to be sprayed tangentially to turbulence chamber 3 towards the center of the circular ring. Put another way, the circular ring connects conduits 512 to the inlet of turbulence channels 4, 41, 42, 43. Said turbulence channels 4, 41, 42, 43 convey the fluid to the truncated cone turbulence chamber 3 tangentially to the cone to create a turbulence.

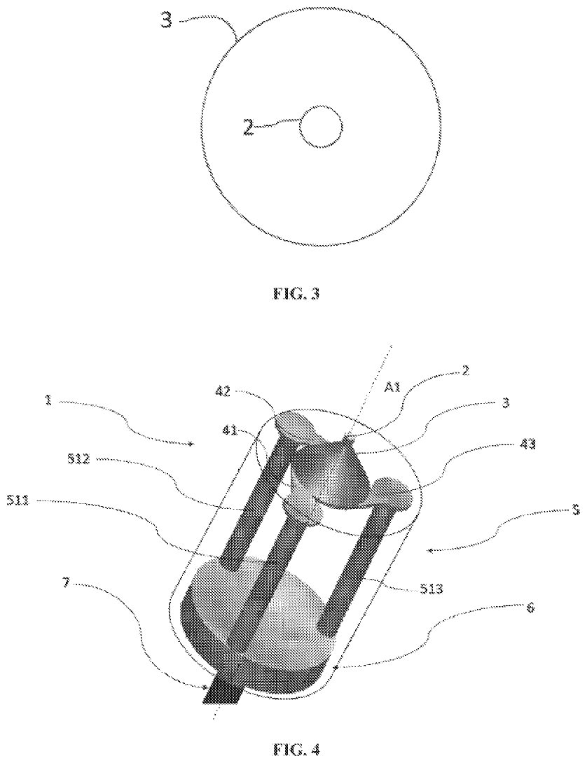

FIG. 3 shows a front view of spray orifice 2 and turbulence chamber 3. In the context of the invention, the ratio of the cross-sectional area s of the spray orifice to the maximum cross-sectional area S of the turbulence chamber is such that 1%≤^{S}/S≤20% and preferably, this ratio is between 1 and 10%, even more preferably, this ratio is between 1 and 6%. It should be noted that respective limits of these intervals are included in the invention.

A circular ring, visible in FIG. 7, connects conduits 511, 512, and 513 to the three turbulence channels 41, 42, and 43 with which they are respectively in fluid connection. The three turbulence channels 41, 42, and 43 each have a rectangular cross-section. This cross-section is between 0.001 and 0.06 mm², preferably between 0.003 and 0.01 mm². This section makes it possible to:
to further increase the shear rate of the fluid and thus further reduce the viscosity,
to increase the fluid velocity (acceleration) in relation to the fluid velocity in the upstream channels, thus having a high velocity on arrival in the vortex chamber 3 to create a faster vortex and thus a better spray,
to have a high fluid velocity, allowing a spray to be generated, at a relatively low flow rate (flow rate=velocity×section, at a given velocity, the lower the section, the lower the flow rate).

As with inlet capillary 7, reducing the cross-section of turbulence channels 41, 42 and 43 increases the shear of the fluid and thus its speed. This increase in speed allows for better turbulence generation and therefore better spraying.

The length of turbulence channels 41, 42 and 43, i.e., the distance to be covered by the fluid to be sprayed between the circular ring and the tangential inlet of turbulence chamber 3 is ideally between 0.2 and 0.71 mm.

FIG. 4 also shows turbulence chamber 3, which has a truncated cone shape with a base diameter ideally between 0.8 and 1.6 mm.

Preferably, angle α between axis A1 and the generatrix of the truncated cone-shaped chamber is such that 25°≤α≤55°, preferably: 30°≤α≤45°.

The height L3 of the truncated cone-shaped chamber is ideally between 0.4 and 0.7 mm.

Finally, in FIG. 4, we also see spray orifice 2, which has a cylindrical shape with a diameter d preferably between 0.05 mm and 0.5 mm, preferably between 0.1 mm and 0.18 mm.

The height h of the spray orifice 2 is ideally between 0.1 mm and 0.15 mm.

FIG. 5 shows a second embodiment according to the invention. In this termination mode, all the elements and their dimensions given for the first embodiment are identical except for the supply means 6, which is here a set of three angularly equidistant supply channels 61, 62, and 63 in fluid connection with conduits 511, 512 and 513. This embodiment makes better routing of the fluid possible from inlet capillary 7 to conduits 51, 511, 512, 513.

FIG. 13 shows an alternative embodiment of this invention. FIG. 13 thus illustrates the fluid path followed by a fluid to be expelled in the form of a mist through a nozzle 1 such as the one shown in FIG. 11 with the inlet capillaries formed by the spacer in FIG. 12. The fluid path downstream from the supply means 6 is identical to those described for the embodiments of FIGS. 7, 8, and 9.

According to some embodiments, nozzle 1 according to the invention may be considered entirely as a consumable and is therefore made of disposable and/or very short-lived materials. Nozzle 1 according to this invention is thus adaptable to many applications in cosmetics, food processing, and is therefore not limited to the medical field.

Nozzle 1 is used in combination with an independent actuator. Spray nozzle 1 is thus actuated by means of an actuator that is independent of the nozzle. “Nozzle actuation” means “circulation of the fluid to be dispensed through nozzle 1”.

This independent actuator can take many different forms, but in all cases it includes a means of circulating the fluid to be sprayed. The actuator may be manual or automated using a mechanical system (pump, syringe pump, spring) or electromechanical (using a motor). The choice of the actuator and the means of circulation of the fluid to be sprayed depends on the desired properties of the spray: size of the cone, flow rate, duration of the spray, for example.

From the foregoing, it is apparent that nozzle 1 according to the invention makes a high shear of a rheofluidifying viscous fluid possible so as to be able to spray this type of fluid effectively and safely. The diameter of the turbulence channels 41, 42, and 43 is small enough to spray a low flow rate mist, but large enough not to induce excessive pressure drops so as to minimize inlet pressure of nozzle 1.

FIG. 10 shows the rheogram (viscosity vs. shear rate curve) of a fluid that has been sprayed with nozzle 1.

Nozzle 1 according to the invention thus allows the implementation of a process for dispensing a rheofluidifying viscous fluid by spraying. More specifically, this distribution is performed in the form of a mist with homogeneous drops whose characterization by Laser Diffraction (Spraytec/MAL10332887/Malvern/UK) makes it possible to establish the following characteristics:
at least 90% of the drops in the mist have a diameter of less than 100 μm, preferably less than 90 μm, more preferably less than 80 μm, even more preferably 70 μm. In a last preferred mode, less than 60 μm, in other words, that the mist has a Dv90 less than 100 μm,
the median diameter of the mist drops, also referred to as the Dv50 of the mist is between 10 and 50 μm, preferably between 10 and 45 μm, more preferably between 15 and 40 μm,
12% of the drops have a diameter of less than 10 μm, preferably less than 10 μm,
a distribution of the different drop sizes of a mist concentrated around its median value (Dv50), such that the ratio between the difference between Dv90 and Dv10, and Dv50 is less than 2, preferably less than 1.8, more preferably less than 1.6. In other words, the “SPAN” distribution is less than 2, preferably less than 1.8, more preferably less than 1.6.

### REFERENCED NUMBERS

1: Spray nozzle
2: Spray orifice
3: Turbulence chamber
4, 41, 42, 43: Turbulence channels
5: Pillar 51, 511, 512, 513: conduits
52: enveloping cylinder
53: Spacer
6: Supply means,
61,62,63: supply channels
7: inlet capillary(ies)
8: Support
81: First release of the support capable of receiving the container of liquid to be dispensed,
81: second release of the support capable of receiving the grooves forming inlet capillaries,
71,72, 73, 74: portions of constant section of inlet capillary 7
A1: axis of the spray orifice
H1, H2: height of the pillar
D7: diameter of the fluid inlet capillary
h7: radial distance between the axis of the spray orifice and the axis of the fluid inlet capillary.
L: length of the fluid inlet capillary
d: diameter of the spray orifice
h: height of the spray orifice
L3: Height of the vortex chamber (according to the axis A1)
α: angle between the axis A1 and the generatrix of the turbulence chamber.

1. A nozzle for spraying a fluid, the nozzle being designed to be mounted on a dispensing container, said nozzle comprising:
at least one fluid inlet capillary extending longitudinally along an axis A1,
a turbulence chamber for receiving the fluid to be sprayed, said turbulence chamber has a maximum cross-section S and a maximum diameter-D,
at least two conduits that extend longitudinally along said axis and being radially offset from said axis, said conduits in fluid connection with the inlet capillary,
at least two turbulence channels, in fluid connection with said at least two conduits, and connecting said at least two conduits with the turbulence chamber, said at least two conduits thus connecting the inlet capillary to the at least two turbulence channels,
a spray orifice supplied by the turbulence chamber, the spray orifice has axial symmetry and a constant cross-section s, said turbulence chamber has along the axis, a cross-section that decreases towards said spray orifice,
wherein
the ratio of the cross-sectional area s of the spray orifice to the maximum cross-sectional area S of the turbulence field is such that 1%≤^{S}/S≤20%,
the spray nozzle is operated by means of an actuator independent of the nozzle, and
the inlet capillary has a cross-sectional area that allows a fluid shear rate greater than 5000 s⁻¹.

2. The spray nozzle (1) according to claim 1, wherein 1%≤^{S}/S≤10%.

3. The spray nozzle according to claim 1, wherein the spray orifice has a cylindrical shape with a diameter d and a height h such that: 40% d≤h≤150% d.

4. The spray nozzle according to claim 1, wherein the at least two turbulence channels each have a right-angled quadrilateral cross-section, said cross-section being between 0.001 and 0.06 mm².

5. The spray nozzle according to claim 4, wherein the quadrilateral is a square.

6. The spray nozzle according to claim 1, wherein the supply means comprises
either a hollow section chamber of generally cylindrical shape, the base of which extends along a plane perpendicular to the axis,
or several supply channels that extend radially on a plane perpendicular to the axis,
in order to supply said at least two conduits.

7. The spray nozzle according to claim 1, wherein the turbulence chamber has a truncated cone shape whose angle α between the axis and the generatrix is such that 25°≤α≤55°.

8. The spray nozzle according to claim 1, wherein the at least two conduits are provided in a pillar, said pillar comprising an enveloping cylinder and having an inner surface Sc, said enveloping cylinder comprising a coaxial spacer whose outer surface is polygonal such that the edges of the spacer are in contact with the inner surface of the enveloping cylinder thereby forming at least three conduits in the pillar.

9. The spray nozzle according to claim 1, wherein the at least one inlet capillary comprises at least two portions each with a constant diameter along its length, each portion with a diameter D equal to or greater than the diameter of at least one downstream portion and each portion with a diameter equal to or less than the diameter of at least one upstream portion.

10. A medical device suitable for dispensing a fluid and comprising a nozzle according to claim 1.

11. A method of dispensing a rheofluidifying viscous fluid by spraying, wherein the method comprises using the nozzle according to claim 1.

12. The method according to claim 11, wherein the distribution is carried out in the form of a mist with homogeneous drops, at least 90% of the drops of the mist having a diameter of less than 100 μm.

13. The method according to claim 12, wherein the distribution is carried out in the form of a mist with homogeneous drops whose median diameter is between 10 μm and 50 μm.

14. The method according to claim 12, wherein the distribution is carried out in the form of a mist with homogeneous drops of which less than 12% of the drops have a diameter of less than 10 μm.

15. The method according to claim 11, wherein the distribution is carried out in the form of a homogeneous mist, the dispersion of the drops of which, characterized by the ratio of the deviation of Dv10 and Dv90 from the median, is less than 2.

16. The spray nozzle according to claim 7, wherein the turbulence chamber has a truncated cone shape whose angle α between the axis and the generatrix is such that 30°≤α≤45°.
